(19) United States
(12) Patent Application Publication
(10) Pub. No.: US 2012/0101363 A1
(43) Pub. Date: Apr. 26, 2012

(54) METHODS AND APPARATUS FOR PATIENT TREATMENT USING MAGNETIC MEDICAL HARDWARE

(76) Inventors: Andrew C. Gordon, Chicago, IL (US); Andrew C. Larson, Kildeer, IL (US); Reed A. Omary, Wilmette, IL (US)

(21) Appl. No.: 13/272,857

(22) Filed: Oct. 13, 2011

Related U.S. Application Data
(60) Provisional application No. 61/392,757, filed on Oct. 13, 2010.

(51) Int. Cl. A61N 2/00; A61B 5/055; A61B 17/03

(52) U.S. Cl. 600/411; 600/9

(57) ABSTRACT

Methods, systems, and apparatus are disclosed to provide treatment to patient tissue using medical hardware incorporating magnetic material. Methods, systems, and apparatus are disclosed to provide magnetic induction heating of cells at a tissue site in a patient.

### CROSS-REFERENCE TO RELATED APPLICATIONS

The present application claims the benefit of priority to U.S. Provisional Application No. 61/392,757, filed on Oct. 13, 2010, entitled “Methods and Apparatus for Patient Treatment Using Magnetic Medical Hardware”, which is herein incorporated by reference in its entirety.

### FIELD OF THE DISCLOSURE

This disclosure relates generally to medical hardware incorporating magnetic material, and, more particularly, to providing treatment to patient tissue using medical hardware incorporating magnetic material.

### BACKGROUND

In recent years, surgical procedures, such as the Cox-Maze procedure, require extensive surgery including cardiopulmonary bypass to treat atrial fibrillation and/or other disorders. Such procedures are time-consuming, involve risk, and often lead to uncomfortable and prolonged healing processes for patients.

### BRIEF DESCRIPTION OF THE DRAWINGS

FIG. 1 shows an example positioning of surgical hardware made and/or treated using magnetic material.

FIG. 2 illustrates an example region of a patient organ after ablation using an alternating magnetic field and magnetic sutures.

FIG. 3 is an example system for magnetic ablation/hyperthermia of patient tissue.

FIG. 4 depicts a flow diagram for an example method for magnetic ablation/hyperthermia treatment of a patient.

FIG. 5 is a block diagram of an example computer or other processor system that can be used to implement systems, apparatus, and methods described herein.

As used in this patent, stating that any part (e.g., a component, module, subsystem, device, controller, generator, hardware, imager, etc.) is in any way positioned on (e.g., positioned on, located on, disposed on, or formed on, etc.) another part, means that the referenced part is either in contact with the other part, or that the referenced part is above the other part with one or more intermediate part(s) located therebetween. Stating that any part is in contact with another part means that there is no intermediate part between the two parts.

### DETAILED DESCRIPTION

Magnetic materials can be incorporated into the composition of medical hardware, such as sutures, wires, clips, staples, and/or other surgical hardware, to permit heating of local tissue. The hardware (e.g., suture, wire, clip, staple, etc.) can be coated and/or externally labeled with magnetic material and/or magnetic material can be incorporated into the internal composition of the hardware (e.g., iron oxide can be integrated with metallic fibers forming medical sutures). Once the medical hardware with the magnetic material is sewn/secured into to a tissue of interest (e.g., a myocardium), a patient can receive local hyperthermia or ablation treatments.

For example, tissue ablation can be performed using the magnetic medical hardware by positioning a tissue area of interest within a magnetic field, such as a rapidly switching magnetic field. After sewing/securing in the material, the ablation heating procedure can be performed one or more times to enhance therapy. The magnetic material can be implanted permanently or temporarily place for removal after therapy. Furthermore, a rotating alternating magnetic field applied can be applied to increase uniformity of ablation therapy.

In certain examples, magnetic induction heating procedures can be used to heat tissue local to the position of magnetic resonance imaging (MRI) visible hardware such as sutures, wires, clips, and/or staples. The hardware incorporates a material such as a ferromagnetic, superparamagnetic, and/or paramagnetic material to permit magnetic inductive heating of the tissue near the hardware. Magnetic material can be incorporated into medical hardware via labeling, exterior coating, and/or as part of the internal composition of the hardware, for example. In some examples, magnetic hardware can serve as a contrast agent for enhanced magnetic resonance (MR) imaging visibility. Magnetic inductive heating can be accomplished using a coil, for example, positioned near tissue of interest including the hardware with ferromagnetic, superparamagnetic, and/or paramagnetic material. A radiofrequency electrical power source sends an alternating current through the coil. Providing an alternating current through the coil generates an alternating magnetic field that causes the therapeutic magnetic material to heat surrounding tissue thereby causing a selected treatment, such as local hyperthermia or ablation. For example, local hyperthermia or ablation procedures can be accomplished with magnetically functionalized sutures, wires, clips, and/or staples.

Using magnetic medical hardware, a surgeon can complete a full Cox-Maze procedure without a cardiopulmonary bypass. Using magnetized hardware, time involved in the Cox-Maze procedure can be reduced. In an example, a magnetic suture allows MR imaging of the suture line for post operative evaluation and subsequent treatment planning Certain examples allow for potential treatment for occurrence of pannus formation in mechanical and/or bioprosthetic heart valves. Certain examples improve completeness of ablation procedures. Certain examples potentially enhance a rate of tissue healing along suture lines. Safety of ablation and hyperthermia treatments can be enhanced through careful manipulation of the material's Curie point. Additionally, magnetic clips and staples can be used as superior anastomotic devices and/or other application(s). Certain examples allow for targeted delivery of therapeutic agents/drugs to a suture line by exploiting magnetic targeting methods.

For example, some studies suggest that occlusion of the left atrial appendage may be associated with a reduced occurrence of thrombolytic events. Methods for occlusion of the left atrial appendage vary and have had limited success. Magnetic sutures allow for sewn isolation of the left atrial appendage followed by ablation for complete occlusion.

The use of sutures to deliver localized hyperthermia or ablation provides significant advantages over similar therapies involving magnetic nanoparticles and/or microspheres. While distribution of such particles cannot be controlled or predetermined, distribution and spacing of magnetic sutures can be determined by a surgeon. Therefore, the surgeon can precisely control the distribution of heating delivered via magnetic sutures exposed to an alternating magnetic field by controlling the distribution of the actual sutures.

Multiple loco-regional oncologic therapies involve injection and/or implantation of radioactive materials (e.g., beads, microspheres, seeds, etc.) to provide locally high doses of activity to malignant tissues while reducing or minimizing radiation exposure to normal surrounding tissues. The overall sensitivity of various bodily tissues to ionizing radiation is dependent upon multiple physiologic factors. Tissue hyperthermia has been shown to increase radiosensitivity. Incorporation of magnetic materials into the composition of sutures, wires, clips, and/or staples, for example, permits heating of local tissue to enhance radiosensitivity.

Using medical hardware, such as wire, suture, clip, and/or staple composition, that is MRI imageable as well as allowing for applications in hyperthermia, multiple independent therapies can be applied to achieve enhanced treatment of targeted tissues. The implementation of a wire, suture, clip, and/or staple, for example, to deliver a local magnetic susceptibility can provide vast advantages over previously investigated techniques. First, MRI mapping of magnetic distribution using such a system can serve as a substitute for mapping distribution of scar tissue in ablated regions. Second, a strong applied magnetic field can be used to localize particles within a desired treatment region. Third, manipulating the Curie point of such materials can serve as a safety mechanism by limiting temperatures generated when exposed to alternating magnetic fields. Therapy can be delivered via specialized wires, sutures, clips, and/or staples that can be either permanently implanted or removed post-therapy, for example.

### BRIEF DESCRIPTION

Certain examples provide a method for magnetic induction heating of cells at a tissue site in a patient. The method includes providing a trigger, via a controller, to control a field generator. The method also includes inducing, using the field generator, a magnetic field with respect to a magnetic surgical hardware positioned at a tissue site for heating. The method includes monitoring the magnetic field and heating at the tissue site with respect to the surgical hardware to evaluate fusion of tissue at the tissue site.

Certain examples provide a system for magnetic induction heating of cells at a tissue site in a patient. The system includes a controller to provide a trigger to control a field generator. The system includes a field generator to induce a magnetic field with respect to a magnetic surgical hardware positioned at a tissue site for heating. The controller is to monitor the magnetic field and heating at the tissue site with respect to the surgical hardware to evaluate fusion of tissue at the tissue site.

Certain examples provide a method for magnetic induction heating. The method includes facilitating application, using a power source, of an alternating current to a coil positioned near a tissue site of interest for a patient. The method includes enabling heating of the tissue at the tissue site through a magnetic field generated by the current in the coil with respect to a magnetic surgical material at the tissue site to form scar tissue at the tissue site.

The Cox-Maze Procedure

According to the American Heart Association, roughly 2.2 million Americans have atrial fibrillation. The “cut and sew” Cox-Maze procedure is widely considered the most well-established and successful intervention for the treatment of atrial fibrillation. This procedure involves the creation of scar tissue to form a maze that directs the conduction pathway in the heart and blocks alternate pathological pathways. Due to the great length of the full “cut and sew” procedure and the need for cardiopulmonary bypass during the operation, many cardiothoracic surgeons choose to perform an abbreviated version of the procedure as a left- or right-sided Maze when the age of the patient or other factors become a consideration. Many surgeons also choose to simply isolate the pulmonary veins in the treatment of atrial fibrillation. Furthermore, techniques such as irrigated monopolar radiofrequency ablation, bipolar radiofrequency ablation, high-intensity-focused ultrasonography, laser, microwave, and cryothermia have been recently investigated as methods to quickly accomplish the Maze procedure. Unfortunately, these techniques remain inferior to the original “cut and sew” method due to incomplete scar tissue formation and difficulty in ablation of the isthmus near the mitral valve annulus.

Currently, the Maze procedure is recommended for all atrial fibrillation patients undergoing cardiac surgery. In addition, the Maze procedure is being investigated as a stand-alone surgery for the treatment of atrial fibrillation.

Using wires and/or sutures made from a ferromagnetic, superparamagnetic, or paramagnetic material, ablation can be used to perform a full or complete Cox-Maze procedure. Placement of wires and/or sutures is the same as with the “cut and sew” method. However, the sutures can be placed free from cardiopulmonary bypass. When the sutures are in place, application of alternating magnetic fields results in heating of tissues adjacent to the suture line to cause ablation. If the ablation is incomplete, the alternating magnetic field (AMF) exposure can be delivered additional times. As a result, a complete “off-pump” Maze procedure is performed in less time than the original “cut and sew” operation. MR imaging can be used to postoperatively evaluate the intervention, for example. This may increase the feasibility of performing the Maze procedure in older patients and patients who currently are not ideal candidates for the complete “cut and sew” procedure.

Roughly 40% of patients receiving the Maze procedure require a postoperative pacemaker. In these patients, AMF exposure can occur prior to pacemaker implantation since the cardiac pacemaker is a contraindication for AMF exposure.

Occlusion of the Left Atrial Appendage

Atrial fibrillation patients have an increased risk of stroke, and it is believed that the thromboembolic origin for strokes in these patients generally comes from the left atrial appendage. Therefore, various methods have been investigated to occlude the left atrial appendage with clips, bands, and/or closure devices such as the Watchman. In addition, excision as well as stapler and suture exclusion can also be used for occlusion of the left atrial appendage. Unfortunately, unsuccessful closure of the left atrial appendage is common with these prior methods.

Magnetic hardware, such as ferromagnetic, superparamagnetic, and/or paramagnetic wires, sutures, clips, and/or staples can be used for exclusion of the left atrial appendage. Subsequent ablation can be performed for complete occlusion. In addition, the suture line, for example, can be MRI visible and can potentially allow for improved postoperative evaluation of the intervention.

Therapy for Pannus Formation

Pannus formation involves the growth of tissue including myofibroblasts, fibroblasts, and capillary endothelial cells in response to surgical injury. Mechanical heart valve prosthesis as well as bioprosthetic heart valves can be adversely affected by significant pannus formation. In bioprosthetic valves, the onset of pannus may thicken the cusps of the valve and the pannus tissue may calcify. Furthermore, calcification near the commissure to the cusp may result in an altered stress profile at the interface between the normal tissue and the calcified tissue that can result in tears. Pannus formation has been found to be the second most frequent indication for reoperation for valve dysfunction in one study. However, there exists some variance in the literature in the estimated frequency of pannus versus thrombosis and, thus, remains an area of debate.

Securing mechanical or bioprosthetic heart valves in place in a patient with ferromagnetic, superparamagnetic, or paramagnetic wires or sutures allows for delivery of magnetic hyperthermia for treatment of pannus formation. Magnetic hyperthermia provides a noninvasive method that can potentially eliminate a need for reoperation. Furthermore, treatments can be delivered as many times as necessary, and the valve ring will remain visible with MRI techniques despite occurrence of pannus overgrowth.

Targeted Delivery of Therapeutic Agents

Magnetic nanoparticles can be used for magnetic targeting. An application of an external magnetic field to a site of interest can be used to hone magnetic nanoparticles to the site of interest. Nanoparticles can provide a vast array of functions in MR imaging, magnetic hyperthermia, and drug delivery. A ferromagnetic, superparamagnetic, or paramagnetic wire, suture, clip, or staple can be used to hone magnetic nanoparticles to the wire, suture, clip, or staple. Using a magnetic field, therapeutic agents can be directed to a targeted tissue of interest. Therapeutic agents can be magnetic and/or delivered via a magnetic carrier such as a magnetoliposome. Furthermore, release of the therapeutic agents can be subsequently accomplished in an actuated method where an AMF is applied to trigger release from a thermo-sensitive carrier, for example.

Enhanced Tissue Healing

Use of microwave diathermy to induce mild hyperthermia (e.g., a temperature of 41-45° C.) has a therapeutic effect in short-term management of musculoskeletal injuries, for example. Hyperthermia can increase metabolic activity thereby increasing oxygen and nutrients at a site of injury. Hyperthermia can also relieve pain, assist in waste removal, increase perfusion, increase cytokine production for stimulation of repair mechanisms, and/or reduce muscle and joint stiffness while increasing tendon extensibility, for example. Thus, certain examples use ferromagnetic, superparamagnetic, and/or paramagnetic medical hardware including wires, sutures, clips, and/or staples to deliver hyperthermia to a tissue and enhance a rate of tissue healing.

Anastomotic Devices

In certain examples, magnetic medical hardware such as ferromagnetic, superparamagnetic, and/or paramagnetic wires, sutures, clips, and/or staples can be used to form anastomotic devices used to create a surgical connection between separate and/or severed tubular organs to form a channel, such as between parts of an intestine.

Oncologic Applications

Magnetic nanoparticles, used intracellularly and/or extracellularly, can be used to treat tumors with electromagnetic hyperthermia. Electromagnetic hyperthermia involves heating magnetic nanoparticles taken up or absorbed by a tumor with an alternating magnetic field (AMF) to achieve temperatures above 43° C. where cell death occurs in cancerous tissues. More recently, hyperthermia has been shown to enhance radiation therapies. Using specialized hardware, such as wires, sutures, clips, and/or staples treated with magnetic material, tumors can be treated and/or imaged with magnetic hyperthermia.

Curie Point for Specifically Selecting Therapeutic Temperatures

In certain examples, a Curie point for a specially designed material can be modified such that an amount of electromagnetic energy absorbed by the material decreases significantly after a specific threshold temperature (e.g., the Curie point) has been reached. Therefore, materials designed in this way can be used to limit a rate at which electromagnetic energy is absorbed at certain temperatures. Manipulation of the Curie point can serve as a safety mechanism that defines a steady-state condition of maximum or threshold temperature produced by a material independent of the field strength(s) used to generate inductive heating in that material.

By implementing techniques to specify the Curie point of ferromagnetic, superparamagnetic, and/or paramagnetic wires and/or sutures, for example, a specific therapeutic temperature can be designated prior to treatment. The Curie point temperature is independent of the field strength of the AMF. Such a safety mechanism can help prevent overheating of normal tissues.

Rotating AMF

Creation of uniform static magnetic fields involves ongoing maintenance (e.g., shimming) in a typical MRI magnet. While creation of uniform alternating magnetic fields is an even more challenging task use of rotating alternating magnetic fields can compensate for a lack of uniform rotating alternating magnetic fields. By rotating the alternating fields, global deposition of electromagnetic energy in a material can become more uniform despite inhomogeneities that remain in the rotating plane of the alternating magnetic fields.

Example Ablation/Hyperthermia Procedure

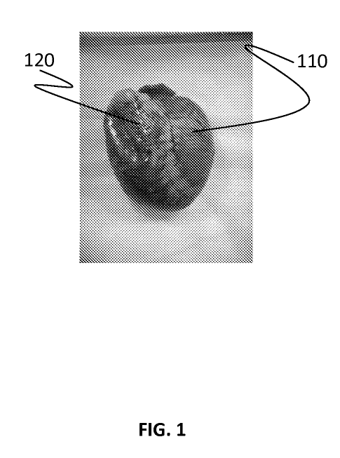

In an example, an ablation/hyperthermia procedure was competed in an explanted rabbit heart for an ex vivo study. A nichrome wire (e.g., 30-gauge (30 G) wire) was fixed to the rabbit epicardium 110 with sutures 120 as shown in FIG. 1 prior to AMF exposure.

An AMF was produced via a 2.4 kW radiofrequency (RF) generator yielding a controlled current through a coil (specified by the operator) alternating at a specific frequency in a 150-400 kHz range. The RF generator has been thoroughly tested and certified to produce consistent, reproducible currents through the specified coil. Current through the coil was chosen based on the desired field strength. Non-ionizing electromagnetic radiation was used in the study, and the frequency range involved in the procedure was the same frequency range used for AM radio broadcasting.

In some examples, AMFs can induce eddy currents in normal tissues that mildly increase the temperature in those tissues. RF induction was chosen for the example application because it can penetrate tissues, such as subcutaneous fat, without excessive heating. The induced electric fields are parallel to the tissue interface meaning that if any heating results from eddy currents, this heating will reside in muscle rather than fat. The pattern of heating generated by the inductive applicator in the example was toroidal in shape with a null at the center of the coil. Therefore, internal organs are less likely to be slightly heated via eddy currents than peripheral tissues. Studies by Atkinson et al. and Stauffer et al. determined that to minimize or reduce these eddy currents, a product of field strength (H) and frequency (f) should satisfy

H·f<4.85×10⁸ A/m·sec

if the diameter of the exposed tissue is 30 cm. A study by Brezovich determined that the deposited power is proportional to this diameter squared. Therefore, assuming that the diameter of the exposed tissue is 3.8 cm, at 200 kHz, the maximum field strength should be 15 kA/m, and, at 400 kHz, the maximum field strength should be 7.5 kA/m. In this example, an appropriate field strength for a given frequency should not be exceeded in clinical applications.

In the example of FIG. 1, the AMF was used to transfer electromagnetic energy to the nichrome wire 120. Samples were placed in a water-cooled Helmholtz-style coil including two 2-turn windings separated by 2.25 inches, each winding with an inner diameter of 2.5 inches and a length of 1 inch. The AMF was applied for 5 minutes in this example.

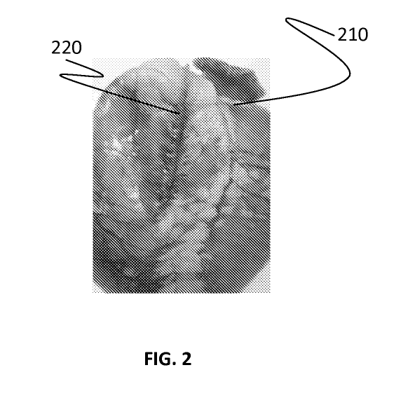

In the example, as depicted in FIG. 2, a controlled ablation at a suture site 220 in a rabbit epicardium 210 was achieved in 5 minutes of AMF exposure. The example study shown in FIG. 2 demonstrates that magnetic nichrome wire can be used to accomplish ablation of heart tissue (e.g., the endocardium, myocardium, pericardium, epicardium, etc.) upon exposure to an AMF.

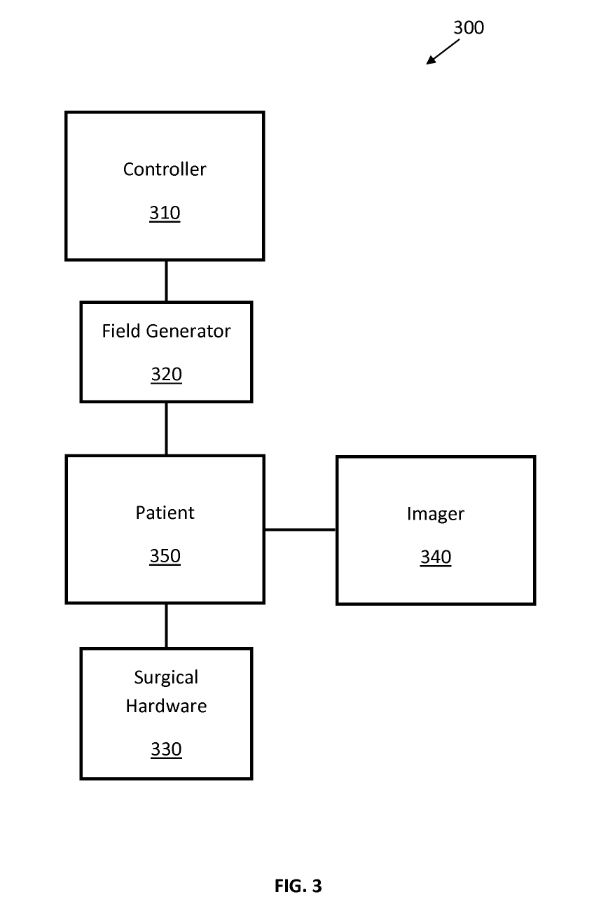

FIG. 3 illustrates an example system 300 for magnetic ablation/hyperthermia of patient tissue. The system 300 includes a controller 310, a field generator 320, surgical hardware 330, and an imager 340 operating with respect to a patient 350.

In the illustrated example of FIG. 3, surgical hardware 330 includes a surgical component such as a wire, suture, clip, and/or staple made of and/or with a magnetic material such as an iron oxide-based magnetic, ferromagnetic, paramagnetic, and/or superparamagnetic material. In the example, the surgical hardware 330 is positioned on and/or in the patient 350. For example, the surgical hardware 330 can be sewn and/or otherwise affixed within the patient 350, such as by stitching a suture along a line between atria of the patient's 350 heart where a scar is desired.

In the illustrated example, the controller 310 controls and triggers the field generator 320, such as an RF current generator including one or more coils. The field generator 320 of the example of FIG. 3 provides a controlled current in the coil(s) to induce a magnetic field with respect to the surgical hardware 330 placed on and/or in the patient 350. In the illustrated example, an alternating magnetic field is generated by the generator 320 to transfer electromagnetic energy to the hardware 330. A user and/or program setting can specify one or more characteristics of the generated field using the controller 310. The controller 310 can be used to set a current, frequency, duration, etc., to generate a desired field strength, size, etc.

The generator 320 of the illustrated example provides electromagnetic energy which heats the material of the surgical hardware 330. The heated hardware 330 of FIG. 3 heat surrounding tissue to form a scar in the tissue. For example, a suture sewn along a line between atria of the patient's 350 heart is heated by an electromagnetic field from the generator 320, which in turn heats tissue within a certain range of the suture hardware 330 to form scar tissue. The scar tissue in this example disrupts a conduction pathway in the intersection between atrial tissue that causes atrial fibrillation. Thus, rather than requiring surgery to cut atrial tissue, sutures and other hardware 330 can be placed along one or more lines in the heart and/or other organ where scar tissue is desired, and the generator 320 provides a magnetic field to the sutures to form scar(s).

In the example of FIG. 3, before, during, and/or after the hardware 330 is inserted in the patient 350 and the generator 320 provides a magnetic field with respect to the hardware 330, the imager 340 can be used to obtain image(s) and associated data to monitor and evaluate results of ablation/hyperthermia applied to patient tissue 350. For example, results can be verified using an MRI. The imager 240 can additionally or alternatively be used to visualize hardware 330 and/or generator 320 coil placement and determine other positioning information for image-guided surgery, image-guided delivery of AMF, etc.

While an example manner of implementing a magnetic ablation system has been illustrated in FIG. 3, one or more of the elements, processes and/or devices illustrated in FIG. 3 can be combined, divided, re-arranged, omitted, eliminated and/or implemented in any other way. Further, the example controller 310, the example field generator 320, the example imager 340, and/or, more generally, the example system 300 of FIG. 3 can be implemented by hardware, software, firmware and/or any combination of hardware, software and/or firmware. Thus, for example, any of the example controller 310, the example field generator 320, the example imager 340, and/or, more generally, the example system 300 of FIG. 3 can be implemented by one or more circuit(s), programmable processor(s), application specific integrated circuit(s) (ASIC(s)), programmable logic device(s) (PLD(s)) and/or field programmable logic device(s) (FPLD(s)), etc. When any of the appended apparatus claims are read to cover a purely software and/or firmware implementation, at least one of the example controller 310, the example field generator 320, or the example imager 340 is hereby expressly defined to include a computer readable medium such as a memory, DVD, CD, Blu-ray, etc., storing the software and/or firmware. Further still, the example system 300 of FIG. 3 can include one or more elements, processes and/or devices in addition to, or instead of, those illustrated in FIG. 3, and/or can include more than one of any or all of the illustrated elements, processes and devices.

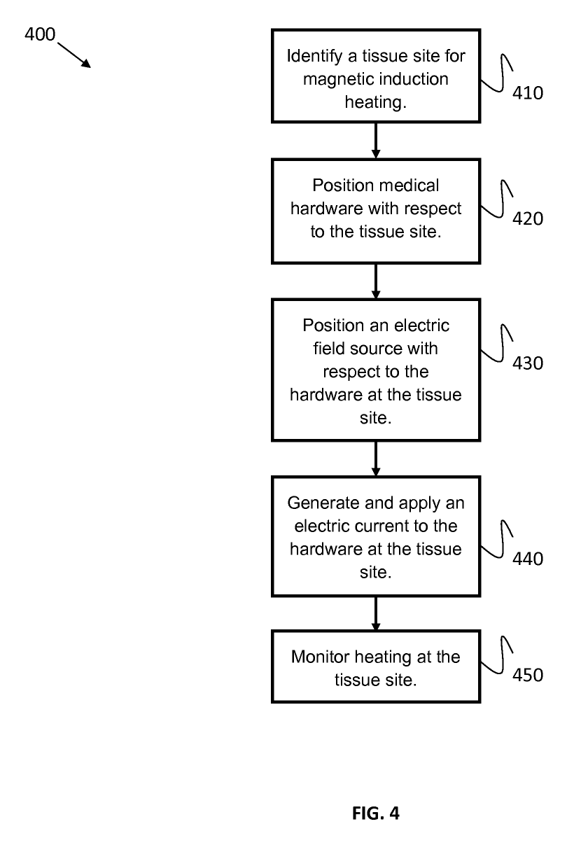

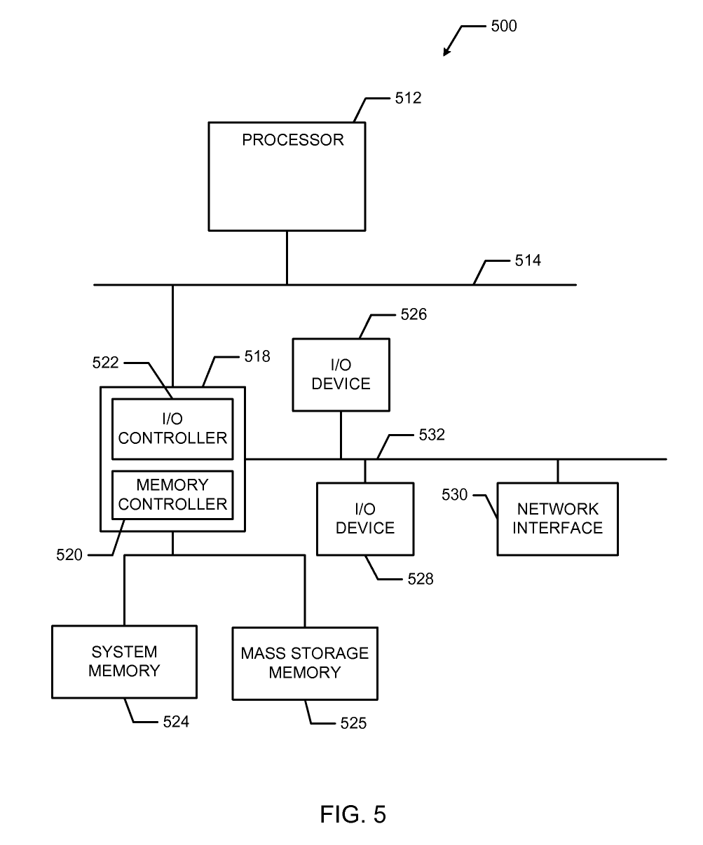

A flowchart including blocks representative of example machine readable instructions for implementing all or part of the system 300 of FIG. 3 is shown in FIG. 4. In this example, the machine readable instructions include a program for execution by a processor such as the processor 512 shown in the example computer 500 discussed below in connection with FIG. 5. The program can be embodied in software stored on a computer readable medium such as a CD-ROM, a floppy disk, a hard drive, a digital versatile disk (DVD), or a memory associated with the processor 512, but the entire program and/or parts thereof could alternatively be executed by a device other than the processor 512 and/or embodied in firmware or dedicated hardware. Further, although the example program is described with reference to the flowchart illustrated in FIG. 4, many other methods of implementing the example system 300 (and/or one or more portions of the system 300) can alternatively be used. For example, the order of execution of the blocks can be changed, and/or some of the blocks described can be changed, eliminated, or combined. Additionally or alternatively, some or all of the method of FIG. 4 can be performed manually by, for example, a surgeon and/or other clinician.

As mentioned above, the example processes of FIG. 4 can be implemented using coded instructions (e.g., computer readable instructions) stored on a tangible computer readable medium such as a hard disk drive, a flash memory, a read-only memory (ROM), a compact disk (CD), a digital versatile disk (DVD), a cache, a random-access memory (RAM) and/or any other storage media in which information is stored for any duration (e.g., for extended time periods, permanently, brief instances, for temporarily buffering, and/or for caching of the information). As used herein, the term tangible computer readable medium is expressly defined to include any type of computer readable storage and to exclude propagating signals. Additionally or alternatively, the example processes of FIG. 4 can be implemented using coded instructions (e.g., computer readable instructions) stored on a non-transitory computer readable medium such as a hard disk drive, a flash memory, a read-only memory, a compact disk, a digital versatile disk, a cache, a random-access memory and/or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

FIG. 4 depicts a flow diagram for an example method 400 for magnetic ablation/hyperthermia treatment of a patient. At block 410, a tissue site for ablation/hyperthermia is identified. For example, a tumor site, a pathological pathway, a cardiac location, and/or other patient tissue site of interest/concern is identified. The site can be identified via image (e.g., an MRI image), biopsy, surgical incision, etc.

At block 420, medical hardware, such as a wire, suture, staple, and/or clip, is positioned and/or enabled to be positioned with respect to the identified tissue site of interest. For example, tissue at the tissue site can be clipped, stapled, and/or sutured using hardware formed from and/or treated with a magnetic material.

At block 430, an electric field source (e.g., a needle or probe electrode including one or more coils and a field generator) is positioned and/or enabled to be positioned with respect to the tissue site. For example, a user, such as a surgeon and/or other clinician, can position the electric field source on and/or in the patient with respect to the tissue site the user wishes to have heated. The electric field source can include and/or be connected to a controller including a user interface and a power unit. The user interface accepts user input and calculates treatment parameters based on the input and possibly other stored information.

At block 440, a current is generated and applied to the medical hardware at the tissue site. The power unit of the field generator generates an electromagnetic field (e.g., an alternating magnetic field) by providing a current to a coil and/or other element based on the treatment parameters, for example. The field introduced in proximity to the medical hardware induces an increase in temperature or heating of the magnetic hardware. Heating of the hardware can be used to generate scar tissue, destroy tumor tissue, increase tissue radiosensitivity for injection and/or implantation of radioactive and/or nanoparticle materials for oncologic therapy, etc.

At block 450, heating at the tissue site is monitored. For example, one or more images, such MRI images, can be obtained to monitor and review results of the tissue heating or ablation. Alternatively or in addition, results can be verified using a biopsy or other examination of a tissue sample at and/or near the target tissue site to determine a resulting state of the tissue (e.g., scarring). Monitoring can also include images taken to visualize hardware and/or field placement and determine other positioning information for image-guided surgery, etc., before, during, and/or after hardware insertion and/or field generation, for example.

FIG. 5 is a block diagram of an example computer or other processor system 500 that can be used to execute one or more of the blocks of FIG. 4 to implement systems, apparatus, and methods described herein, including the controller, generator, and imager of FIG. 4. As shown in FIG. 5, the processor system 500 includes a processor 512 that is coupled to an interconnection bus 514. The processor 512 can be any suitable processor, processing unit, or microprocessor, for example. Although not shown in FIG. 5, the system 500 can be a multi-processor system and, thus, can include one or more additional processors that are identical or similar to the processor 512 and that are communicatively coupled to the interconnection bus 514.

The processor 512 of FIG. 5 is coupled to a chipset 518, which includes a memory controller 520 and an input/output (“I/O”) controller 522. As is well known, a chipset typically provides I/O and memory management functions as well as a plurality of general purpose and/or special purpose registers, timers, etc. that are accessible or used by one or more processors coupled to the chipset 518. The memory controller 520 performs functions that enable the processor 512 (or processors if there are multiple processors) to access a system memory 524 and a mass storage memory 525.

The system memory 524 can include any desired type of volatile and/or non-volatile memory such as, for example, static random access memory (SRAM), dynamic random access memory (DRAM), flash memory, read-only memory (ROM), etc. The mass storage memory 525 can include any desired type of mass storage device including hard disk drives, optical drives, tape storage devices, etc.

The I/O controller 522 performs functions that enable the processor 512 to communicate with peripheral input/output (“I/O”) devices 526 and 528 and a network interface 530 via an I/O bus 532. The I/O devices 526 and 528 can be any desired type of I/O device such as, for example, a keyboard, a video display or monitor, a mouse, etc. The network interface 530 can be, for example, an Ethernet device, an asynchronous transfer mode (“ATM”) device, an 802.11 device, a DSL modem, a cable modem, a cellular modem, etc. that enables the processor system 500 to communicate with another processor system.

While the memory controller 520 and the I/O controller 522 are depicted in FIG. 5 as separate blocks within the chipset 518, the functions performed by these blocks can be integrated within a single semiconductor circuit or can be implemented using two or more separate integrated circuits. The coded instructions of FIG. 4 can be stored in the mass storage device 525, in the system memory 524, and/or on a removable storage medium such as a CD, Blu-ray, or DVD.

From the foregoing, it will appreciate that the above disclosed methods, apparatus, and articles of manufacture providing heating of tissue at a target site using magnetic medical hardware in place of further surgical procedure(s). The above disclosed methods, apparatus, and articles of manufacture provide magnetic induction heating of a localized area within a patient under precise control of a surgeon, for example.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. A method for magnetic induction heating of cells at a tissue site in a patient, the method comprising:
providing a trigger, via a controller, to control a field generator;
inducing, using the field generator, a magnetic field with respect to a magnetic surgical hardware positioned at a tissue site for heating; and
monitoring the magnetic field and heating at the tissue site with respect to the surgical hardware to evaluate fusion of tissue at the tissue site.

2. The method of claim 1, wherein the magnetic surgical hardware comprises a magnetic suture.

3. The method of claim 1, wherein the field generator comprises a radio frequency current generator including one or more coils.

4. The method of claim 3, wherein inducing further comprises inducing, using the field generator, a controlled current in the one or more coils to induce a magnetic field with respect to the surgical hardware placed at least one of on and in a patient at the tissue site.

5. The method of claim 1, wherein the magnetic field comprises an alternating magnetic field generated by the field generator to transfer electromagnetic energy to the surgical hardware.

6. The method of claim 1, further comprising specifying one or more characteristics of the generated magnetic field using the controller.

7. The method of claim 6, wherein the controller is to set at least one of a current, frequency, and duration to generate at least one of a desired field strength and a desired field size.

8. The method of claim 1, further comprising obtaining, using an imager, one or more images and associated data to monitor and evaluate results of heating at the tissue site.

9. A system for magnetic induction heating of cells at a tissue site in a patient, the system comprising:
a controller to provide a trigger to control a field generator; and
a field generator to induce a magnetic field with respect to a magnetic surgical hardware positioned at a tissue site for heating, wherein
the controller is to monitor the magnetic field and heating at the tissue site with respect to the surgical hardware to evaluate fusion of tissue at the tissue site.

10. The system of claim 9, wherein the magnetic surgical hardware comprises a magnetic suture.

11. The system of claim 9, wherein controller is to use the magnetic surgical material as a contrast agent to monitor and evaluate results of heating at the tissue site.

12. The system of claim 9, wherein the field generator comprises a radio frequency current generator including one or more coils.

13. The system of claim 12, wherein the field generator is to induce a controlled current in the one or more coils to induce a magnetic field with respect to the surgical hardware placed at least one of on and in a patient at the tissue site.

14. The system of claim 9, wherein the magnetic field comprises an alternating magnetic field generated by the field generator to transfer electromagnetic energy to the surgical hardware.

15. The system of claim 9, wherein the controller is to specify one or more characteristics of the generated magnetic field.

16. The system of claim 15, wherein the controller is to set at least one of a current, frequency, and duration to generate at least one of a desired field strength and a desired field size.

17. The system of claim 9, further comprising an imager to obtain one or more images and associated data to monitor and evaluate results of heating at the tissue site.

18. A method for magnetic induction heating comprising:
facilitating application, using a power source, of an alternating current to a coil positioned near a tissue site of interest for a patient; and
enabling heating of the tissue at the tissue site through a magnetic field generated by the current in the coil with respect to a magnetic surgical material at the tissue site to form scar tissue at the tissue site.

19. The method of claim 18, wherein the magnetic surgical material comprises at least one of a suture, a clip, a staple, and a wire constructed from at least one of a ferromagnetic, superparamagnetic, and paramagnetic material.

20. The method of claim 18, further comprising obtaining, using a magnetic resonance imager, one or more images and associated data of the tissue site using the magnetic surgical material as a contrast agent to monitor and evaluate results of heating at the tissue site.
